(19) United States
(12) Patent Application Publication
(10) Pub. No.: US 2023/0050058 A1
(43) Pub. Date: Feb. 16, 2023

(54) SYSTEMS AND METHODS FOR NON-INVASIVE PULSE PRESSURE WAVEFORM MEASUREMENT

(71) Applicant: California Institute of Technology, Pasadena, CA (US)

(72) Inventors: Alessio Tamborini, Pasadena, CA (US); Morteza Gharib, Pasadena, CA (US)

(21) Appl. No.: 17/886,374

(22) Filed: Aug. 11, 2022

Related U.S. Application Data
(60) Provisional application No. 63/232,082, filed on Aug. 11, 2021.
(60) Provisional application No. 63/251,762, filed on Oct. 4, 2021.
(60) Provisional application No. 63/253,988, filed on Oct. 8, 2021.
(60) Provisional application No. 63/333,017, filed on Apr. 20, 2022.
(60) Provisional application No. 63/341,113, filed on May 12, 2022.

(51) Int. Cl. A61B 5/021; A61B 5/022

(52) U.S. Cl. CPC A61B 5/02125; A61B 5/02233; A61B 5/02141; A61B 5/02225

(57) ABSTRACT

Systems and methods are provided for a non-invasive high resolution pressure pulse waveform measurement system. The system may include a blood pressure cuff, an air pump to inflate the blood pressure cuff to specific pressure levels, high resolution pressure sensors configured to perform high sensitivity signal acquisition at a specified pressure level, high range pressure sensors configured to measure an absolute reference for the signal and to calibrate the signal, pneumatic tubing connecting the air pump and sensors with the cuff, and a hydrodynamic filter configured as an input to a reference port of the high resolution pressure sensor. The hydrodynamic filter may be configured to transmit only mean pressure by attenuating a selected frequency range of the signal.

### CROSS-REFERENCE TO RELATED APPLICATIONS

This application claims the benefit of U.S. Provisional Application Nos. 63/232,082, filed Aug. 11, 2021, 63/251,762, filed Oct. 4, 2021, 63/253,988, filed Oct. 8, 2021, 63/333,017, filed Apr. 20, 2022, and 63/341,113, filed May 12, 2022, each of which is hereby incorporated herein by reference in its entirety.

### TECHNICAL FIELD

The present disclosure relates generally to medical diagnostics, and in particular, some implementations may relate to non-invasive cardiac waveform measurement.

### BACKGROUND

Heart disease is the leading causing of death for both women and men in the U.S. and worldwide. Early and accurate diagnoses of heart disease may significantly improve patient health outcomes. However, at present, many important cardiovascular health measurements involve invasive, expensive, and/or lengthy procedures. Therefore, it can be difficult to diagnose critical illness, such as heart failure, until advanced disease progression occurs.

Several important measurements allow physicians to diagnose heart failure. The pressure pulse waveform, for example, is an important measurement that allows medical professionals to quantitatively and qualitatively evaluate cardiac health. In typical clinical settings, medical professionals use a handheld force sensor to measure radial pulsations of the artery. A trained operator must perform the measurement to ensure accuracy.

### BRIEF SUMMARY OF THE DISCLOSURE

According to various embodiments of the disclosed technology, a non-invasive pressure pulse waveform measurement system is provided. The non-invasive pressure pulse waveform measurement system may include: a blood pressure cuff; an air pump to inflate the blood pressure cuff to specific pressure levels; high resolution pressure sensors configured to perform high sensitivity signal acquisition at a specified pressure level, wherein each high resolution pressure sensor includes a measurement port and a reference port; high range pressure sensors configured to measure an absolute reference for the signal and to calibrate the signal; pneumatic tubing connecting the air pump and sensors with the cuff; and a hydrodynamic filter configured as an input to the reference port of each high resolution pressure sensor.

In some embodiments, the hydrodynamic filter comprises a resistive component and a capacitive component, wherein the hydrodynamic filter is configured to transmit only mean pressure by attenuating a selected frequency range of the signal. The resistive component of the hydrodynamic filter can be configured to impose a resistance to flow, thereby slowing down the flow. In some cases, the resistive component of the hydrodynamic filter comprises rigid tubing with an internal diameter in the range of 10-200 μm. The capacitive component is configured to reduce pressure changes by storing air volume. In some embodiments, an elasticity of the capacitive element is in the range of 0.2-2.0 MPa. The capacitive component can comprise tubing that connects the resistive component to the reference port.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1 is a diagram showing an example of a modified blood pressure cuff system in accordance with the systems and methods disclosed herein.

FIG. 2A is a diagram showing an example of a resistive component having a fixed orifice in accordance with the systems and methods disclosed herein.

FIG. 2B is a diagram showing an example of a resistive component having an in-line filter in accordance with the systems and methods disclosed herein.

FIG. 2C is a diagram showing an example of a resistive component having a reduced internal diameter in accordance with the systems and methods disclosed herein.

FIG. 2D is a schematic of the capacitive component including an elastic tube.

FIG. 2E is a schematic of the capacitive component including tube with a piston cylinder.

FIG. 3 is a flow diagram showing an example of a pulse pressure waveform measurement method in accordance with the systems and methods disclosed herein.

FIG. 4A is an example plotting of a left ventricle pressure-volume (“PV”) loop in a young patient in accordance with the systems and methods disclosed herein.

FIG. 4B is an example plotting of a left ventricle pressure-volume (“PV”) loop in an old patient in accordance with the systems and methods disclosed herein.

FIG. 5 is a flow diagram showing an example of a left ventricular end-diastolic pressure (“LVEDP”) risk prediction method in accordance with the systems and methods disclosed herein.

FIG. 6 is an example plotting of an envelope function reconstructed at a specified pressure level and three pressure holds in accordance with the systems and methods disclosed herein.

FIG. 7 is an example plotting of an envelope function to estimate systolic BP (“SBP”) and diastolic BP (“DBP”) changes following pulse amplitude fluctuations in accordance with the systems and methods disclosed herein.

The figures are not exhaustive and do not limit the present disclosure to the precise form disclosed.

### DETAILED DESCRIPTION

Embodiments of the systems and methods disclosed herein can provide a modified blood pressure (“BP”) cuff which may be used to perform non-invasive yet accurate cardiac measurements. The systems and methods disclosed herein may be leveraged to perform pressure pulse waveform, left ventricular end-diastolic pressure (“LVEDP”) measurements, pressure-volume (“PV”) loop measurements, and other important cardiac measurements. The systems and methods herein related to a modified BP cuff system, methods for performing measurements, risk assessment measurements, and calibration methods.

### Non-Invasive Pulse Pressure Waveform Measurement

A modified blood pressure (“BP”) cuff may be used to measure a pressure pulse waveform. In some embodiments, a modified BP cuff system may include a dynamic pressure sensor instead of and/or in addition to a static pressure sensor. A high resolution pressure sensor may be included for high sensitivity signal acquisition at a specified pressure level. The high resolution pressure sensor may comprise a differential pressure sensor having a measurement port and a reference port, wherein the pressure sensor measures the difference between its measurement port and reference port. A high range absolute pressure sensor may be used to calibrate the signal. An air valve or filter may be included to maintain a specific pressure level at the reference port. Maintaining the pressure level may allow the high resolution pressure sensor to operate within its normal range. During measurement the pressure sensors may simultaneously acquire signals.

The high range pressure sensor may measure with respect to atmospheric pressure while the high resolution pressure sensor may measure with respect to a variable reference pressure. The pressure sensors may be connected in parallel to the BP cuff system. In some embodiments, the high range and high resolution pressure sensors may have an operating range on the order of magnitude of the measurement and signal, respectively.

No control system is employed in the embodiments described herein. Such control systems could be used to dynamically control an air valve to open and close the valve at appropriate pressures to ensure the signal is captured correctly. In such a system, pressure fluctuations may cause sensor saturation resulting in a critical fault, signal drift, and loss of valuable information. For these reasons, an air valve system may present drawbacks that are not present in the embodiments described herein.

In some embodiments, a passive and self-adjusting non-invasive pulse pressure waveform measurement system may include high resolution pressure sensors in an inflatable pressurized air chamber having resistive and capacitive components. Hydraulic filtering may be implemented through a geometrical condition to passively generate a signal that only transits desired frequencies. In some embodiments an air valve may be replaced with a hydrodynamic filter. A hydrodynamic filter may comprise a fixed or adjustable orifice, an in-line filter, and/or tubing with an internal diameter (“ID”) significantly smaller than the ID of the rest of tubing included in a modified BP cuff system.

The hydrodynamic filter can be achieved using a sequential combination of resistive tubing and compliant tubing. The resistive tubing generates a flow resistance limiting the flow that can move across this element. The compliant tubing stores injected volume at a desired compliance rate. This hydraulic system generates the electrical equivalent of an RC low pass filter. In such a hydraulic system, the circuit may be understood as the time the compliant element needs to fill up through the resistive element.

In some embodiments, the hydrodynamic filter comprises a resistive component and a compliant component. The resistive component is configured to impose a resistance to flow, thereby slowing down the flow, whereas the compliant component comprises a capacitive component configured to reduce pressure changes by storing air volume. In such embodiments, the compliant component may comprise tubing that connects the resistive component to the reference port. Additionally, the resistive component of the hydrodynamic filter may comprise rigid tubing with an internal diameter in the range of 10-200 μm. In some cases, an elasticity of the capacitive element is in the range of 0.2-2.0 MPa. The hydrodynamic filter may form an input connection to the reference port and thus regulate flow into the reference port. This configuration may provide the system with steady pressure resulting in a smooth signal.

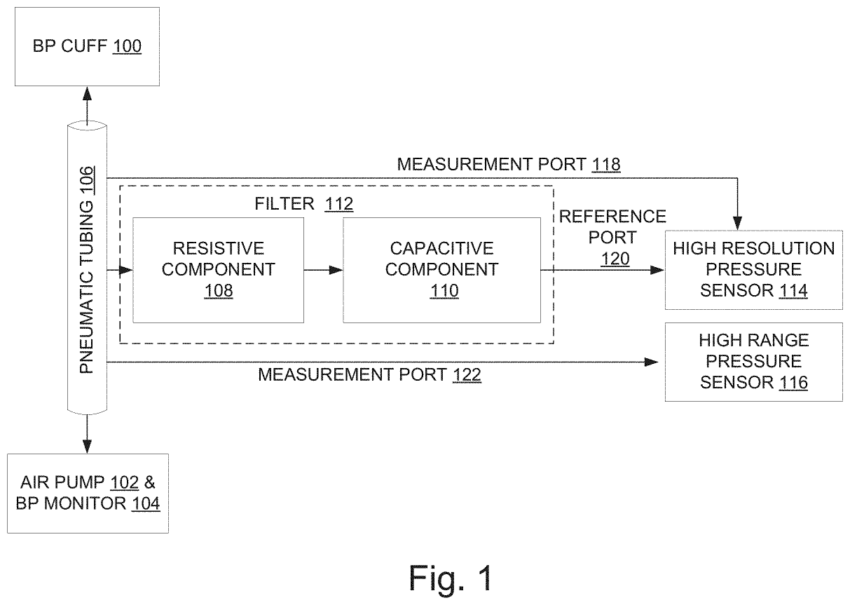

FIG. 1 shows an example of a system having a passive configuration for the high accuracy sensor. As shown in FIG. 1, the system may include a BP cuff 100. The system may also include pneumatic connections 106 for a high resolution pressure sensor 114, a filter 112 including a resistive component 108 in series with a capacitive component 110, and a reference port 120. The system may also include an air pump 102 and a BP monitor 104. The air pump 102 may be used to inflate the cuff 100.

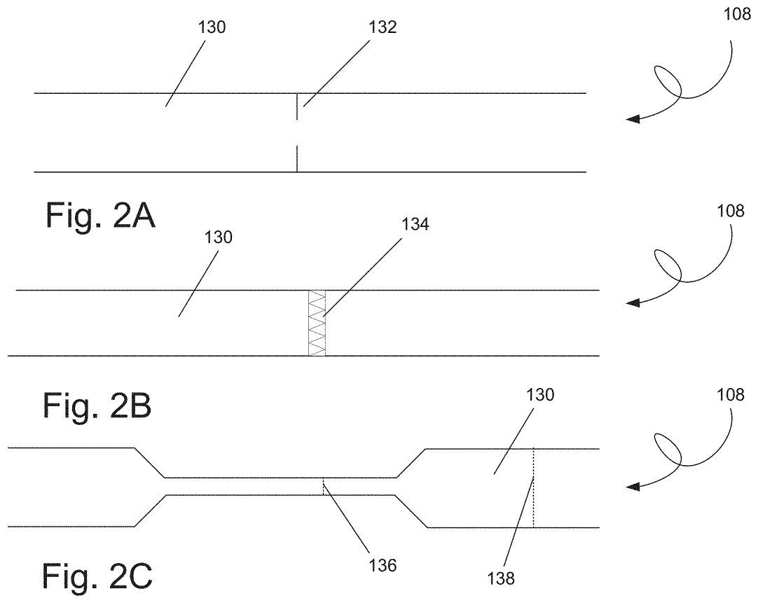

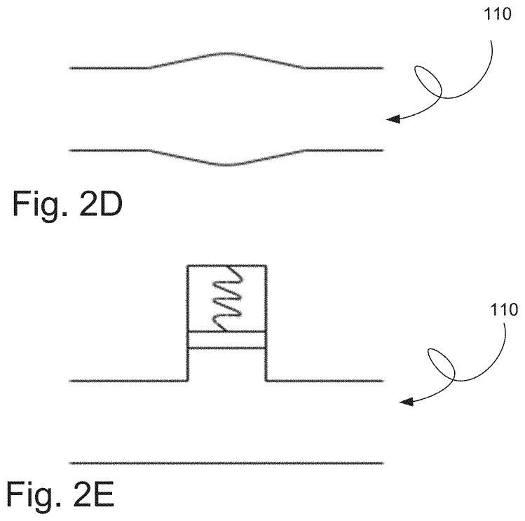

FIGS. 2A, 2B, and 2C are schematics of the resistive component 108 comprising tubing 130 with fixed orifice 132, tube with inline filter 134, and tube with ID 136 smaller than ID 138. As shown in FIG. 2A, the filter 134 may comprise an orifice 132. As shown in FIG. 2B, orifice 132 may be configured in the tubing 130 connecting to the reference port 120. In further embodiments, the orifice may be adjustable. As shown in FIG. 2B, the tubing 130 connecting to the reference port 120 may be configured with an in-line filter 134. As shown in FIG. 2C, the tubing 130 connecting to the reference port 120 may include a portion with a reduced diameter, or a small ID 136 relative to the ID of the tubing 138.

In some embodiments, the filter 134 may comprise a fixed or adjustable orifice 132. The orifice 132 may control the amount of air that can flow between the rest of the BP pressure cuff system and the reference port 120. Air flow across the orifice 132 is driven by a pressure differential. Limiting flow between compartments using an orifice instead of a valve results in smoothing out pressure oscillations while maintaining mean signal, acting as low pass filter on reference port side.

FIGS. 2D and 2E are schematics of the capacitive component 110 including an elastic tube (FIG. 2D), and tube with a piston cylinder (FIG. 2E).

An important measurement may be the difference between the signal measured at the measurement port and at the reference port. Therefore, the output signal is the equivalent of a high pass filtered signal. Additionally, a self-adjusting reference port signal may also maintain a centered output signal. Using an orifice, as opposed to an air valve, allows the reference port to stay at the mean pressure signal. Maintaining the mean pressure signal is important to eliminate bias in the measured pulsations and maintain a centered signal with the high-resolution transducer.

In some embodiments, the hydrodynamic filter may comprise a resistive component comprising a tube with a small ID. In such embodiments, the resistive component may comprise substantially rigid tubing with an internal diameter in the range of 10-200 μm The ID of the tube may be significantly smaller in diameter than the ID pf the tubing connected the rest of the BP pressure cuff system and reference port. The tubing with small ID effectively acts as a filter in which only the mean pressure is transmitted creating a flow dependent low pass filter to the reference port. Specific cutoff frequencies of the filter are designed using fluid dynamic principles and the characteristics of the measuring system and signal. The filtered signal will be dependent on the combination of volumetric flow rate and the material properties of the reference port side.

In some embodiments, the system may be designed using a commercial arm cuff BP system. The system may be modified to include a plurality of pressure sensors with different operating ranges to measure and calibrate the pressure waveform with high accuracy. High resolution pressure sensors may be used for accurate signal measurement. Each High resolution pressure sensor may contain a measurement port and a reference port. High range pressure sensors may be used for absolute reference and signal calibration.

In some embodiments, the system can be applied to any location in the body that has arteries close to the surface and can withstand a brief reduction or cessation of blood flow. Potential locations may include, but are not limited to, brachial, radial, femoral, and posterior tibial.

A pressure sensor may measure peripheral pressure pulse. Per fluid-solid interaction principles, the pressure at which cuff is inflated alters pressure-flow behavior in artery. Combinatorial waveform analysis may then be used to non-invasively assess cardiovascular health. A peripheral pulse waveform may be measured and then may be transformed to estimate the central waveform.

Pressure and flow velocity in closed system may be given by the Bernoulli equation (below). When a brachial cuff is inflated, the externally applied force alters the radius of the artery, ultimately changing the pressure flow proportion of the system. At a lower extreme, applying pressure to the artery below minimum DBP will cause no or minimal alteration to pressure flow behavior. At an upper extreme, pressure above the maximum SBP in the artery causes collapse of artery and cessation of blood flow. Any pressure between these two extremes may create a proportional alteration to the pressure flow relationship, again given by the Bernoulli equation. Comparing a measured waveform at two different hold pressures therefore allows derivation of the pressure- flow characteristics of system. Quantitative and qualitative comparisons of the waveforms may be performed. Plotting methods may also be used such as waveform versus time or waveform versus waveform.

Captured signals reflecting the pressure flow relationship in elastic arteries can be used to derive additional waveforms that may further characterize a patient. Fluid dynamic principles enable deriving waveforms, including, but not limited to, flow, velocity, and radial movement. Fluid-dynamics principles relate parameters such as pressure, velocity, forces, and volumes for static systems. Analyzing these systems with multiple measurement points allows to solve for the interrogated parameters. For example, velocity may be solved for by using DBP and supra SBP hold pressure waveforms. The supra SBP waveform completely obstructs flow, giving an absolute pressure reading. The DBP waveform represents a combination of pressure and flow. Therefore, the resulting flow may be measured during the DBP hold pressure. Similar derivations may be applied to other hold pressure combinations. The significance of the results obtained may depend on the underlying physics of the captured waveform(s).

Practical applications may require synchronization of waveforms. Solutions may include time synchronization of different waveforms using ECG or using known timing events during cardiac cycle including max dP/dt, start, and dicrotic notch. This cuff can have wired or wireless synchronization with other devices, such as Bluetooth or Wi-Fi with ECG

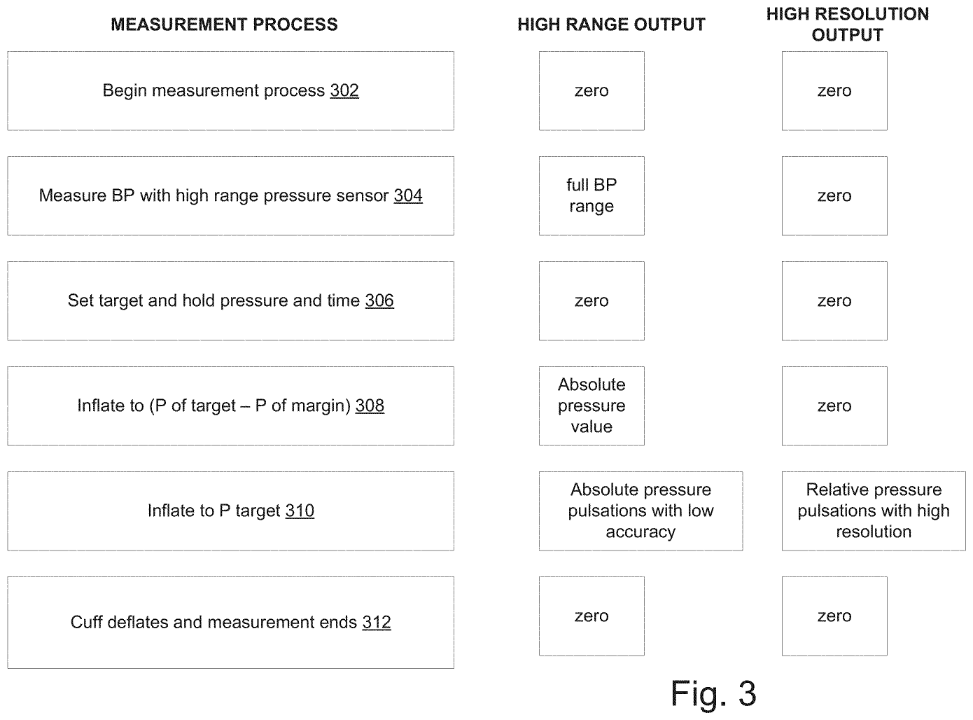

FIG. 3 shows an example of a non-invasive pulse pressure waveform measurement. A modified BP pressure cuff system, as described above, may be used to record pressure pulsations in the brachial artery. To perform the measurement a brachial BP cuff may be inflated around a patient's arm. A pump system may be used to inflate the cuff. A first operation 302 may involve starting the measurement process. At the time of the first operation 302, the output of a high range pressure sensor and a High resolution pressure sensor may both be zero. The pressure sensor may be used to measure the systolic BP (“SBP”) and diastolic BP (“DBP”) of the patient. A second operation 304 may involve measuring BP with a high range pressure sensor. At the time of the second operation 304, the output of a high range sensor may the full BP range for the patient and the output of a high resolution pressure sensor may be zero.

As a third operation 306, target and hold pressure and time may be set. For example, the cuff may be set to be inflated to a pressure of 100 mmHg and may be held for 40 seconds. Other pressures and timing are also possible. At the time of the third operation 306, the output for both the high range and high resolution pressure sensors may be zero. Inflation pressure references and/or targets for the cuff may be obtained by performing traditional blood pressure arm cuff measurements. For example, hold pressures may be set at DBP, below DBP, at SBP, above SBP, and/or at MAP. Typical physiological ranges for these values are as follows: 40-120 mmHg for DBP; 50-150 mmHg for MAP; and 75-225 mmHg for SBP. Other pressures are also possible. For example, extremely sick subjects could have values outside of these ranges. For patient specific values, a specified pressure level may be employed to guide the hold pressure selection. To maintain the high resolution pressure sensor within its operating range, a pressure may applied to the reference port.

As a fourth operation 308, the cuff may be inflated and held at a given pressure. For example, the cuff may be inflated to a pressure of P target, which may be one of the identified hold pressures. At the time of the fourth operation 308, the high range sensor output may be the absolute pressure value and the high resolution pressure output may be zero. As a fifth operation 310, the cuff may then be inflated to the target pressure. At the time of the fifth operation 310, the high range sensor may have an output of the absolute pressure pulsations with low accuracy. The high resolution sensor may have an output of the relative pressure pulsations with high accuracy. As a sixth operation 312, the cuff may deflate, ending the measurement period. At the time of the sixth operation 312, each of the high and high resolution pressures sensors may have an output of zero. For multiple hold pressures, operations three to six may be repeated. Outputs from operation five may be combined for a calibrated high accuracy pulsation output.

In some embodiments, comparisons of waveform captures may be performed using a low hold pressure and a high hold pressure above an upper pressure extreme. For instance, for the low hold pressure, pressure may be set at or just below DBP. For the high hold pressure, pressure may be set above SBP (“supraSBP”), cutting off the flow of blood. For example, pressure may be set at about SBP+35 mmHg. At the lower extreme, the waveform may represent a combination of static pressure and flow velocity. At the upper extreme, waveform only displays pressure characteristics. At supraSBP hold pressures, cessation of flow in subclavian artery becomes closest waveform for representing static pressure read from a hole in the wall of the ascending aorta. This hold pressure allows for direct pressure waveform measurements in central arteries. Respective pressure waveform between supraSBP and DBP can be plotted in pressure-pressure (“PP”) loop for cardiac health and disease assessment. This allows for creation of pressure-velocity (“PV”) loop applied for health assessments.

### Pressure-Velocity (“PV”) Loop Embodiment

As discussed above, flow at the brachial artery may be characterized with the Bernoulli equation for average flow, given by:

P_{B}+1/2ρu_{B}²=P_{T}

where P_{B} is the pressure at the brachial artery, ρ is the fluid density, u_{b} is the flow velocity at the brachial artery, and P_{T} is the total pressure in the aortic arch.

At the supra SBP pressure hold (P_{SS}), the brachial artery is completely occluded resulting in the u_{b}=0. Thus, the measured pressure at the cuff is the pressure in the aortic arch. As such:

P_{SS}=P_{B}=P_{T}

where P_{SS} is the supra SBP hold pressure.

At the DBP pressure hold (P_{D}), the applanation condition measures the pressure in the brachial artery. The pressure in the brachial artery fits in the Bernoulli equation as shown below:

P_{D}=P_{B}→P_{B}+1/2ρu_{B}²=P_{T}

where P_{D} is the DBP hold pressure.

Equating the above through the total aortic arch pressure and solving for the velocity (u_{B}) gives:

$${P}_{SS} = {P}_{D} + \frac{1}{2} ρ {u}_{B}^{2}$$

$${u}_{B} = \sqrt{\frac{2}{ρ} * ( {P}_{SS} - {P}_{D} )}$$

Plotting the supra SBP pressure (P_{SS}) versus the DBP velocity (u_{D}) gives the (“PV”) loop. Data may be plotted and analyzed at any intermediate step. For example, SBP pressure versus DBP pressure may be analyzed.

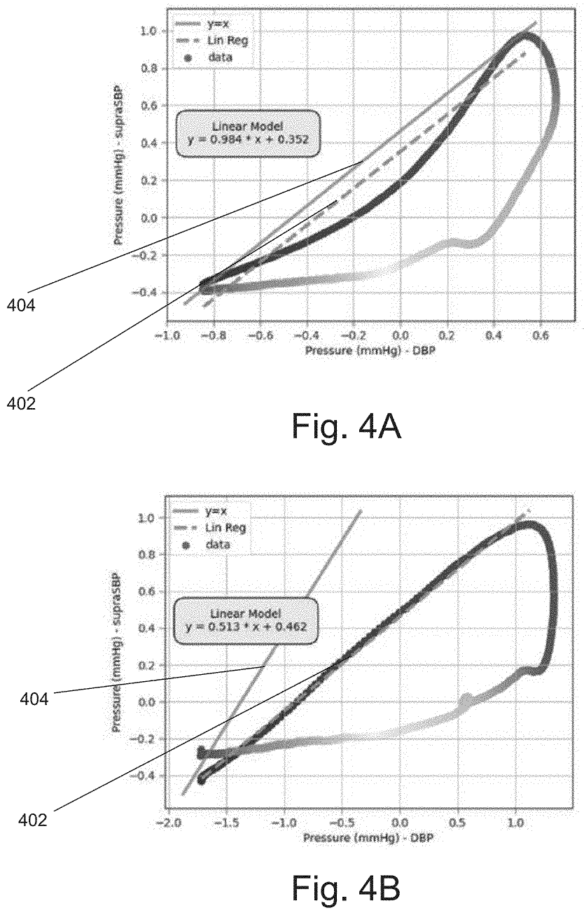

FIG. 4 shows examples of PV loop comparison older (FIG. 4A) and younger (FIG. 4B) patient. Each of the PV loops have different features and shapes, Each includes a dashed line 402 which models the slope of rising systolic portion. The slope can clearly differentiate patients by age, so it is a useful diagnostic. Each also includes a solid line 404 which is a proportionality line. Other parameters include loop areas, curvatures, indentations, peak shifts, and other combinations of parameters.

### Left Ventricular End-diastolic Pressure (“LVEDP”) Risk Prediction Embodiment

LVEDP is an important clinical measurement used to predict, diagnose, and/or assess the risk for heart failure. Currently, the threshold for heart failure is an LVEDP measurement of about 18 mmHg. Because LVEDP is a valuable diagnostic measurement, non-invasive methods for measuring LVEDP may allow clinicians to predict risk of heart failure earlier and with accuracy.

A non-invasive pulse waveform analysis and classification algorithm may be used to form an LVEDP risk prediction.

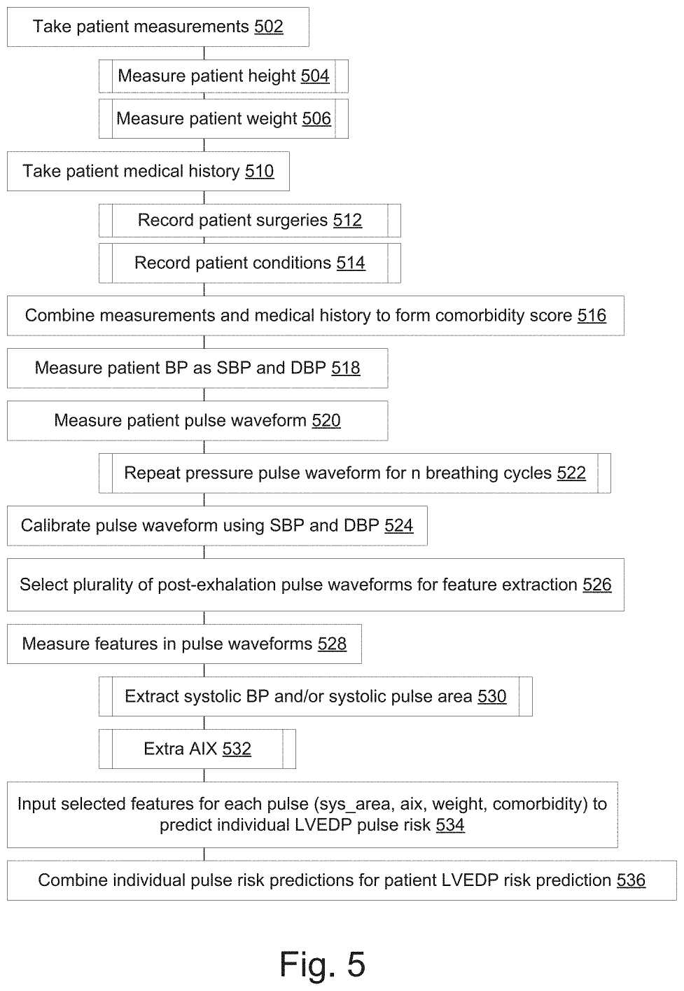

FIG. 5 shows an example of a non-invasive LVEDP risk prediction method 500 involving several operations. A first operation 502 may involve taking patient measurements. A sub operation 504 of the first operation 502 may involve measuring the patient's height. A sub operation 506 of the first operation 502 may involve measuring the patient's weight. Other patient measurements may also be taken. A second operation 508 may involve taking a patient's medical history. A sub operation 510 to the second operation 508 may involve recording patient surgeries. Patient surgeries, such as heart surgeries, may be recorded. Other medical information, such as procedures and other treatments may also be recorded. A sub operation 512 to the second operation 508 may involve recording patient conditions. Patient conditions may include known instances of cardiovascular conditions such as heart failure, myocardial infarction, and cardiomyopathy. Other heart conditions, comorbid conditions, and/or general health conditions may be recorded. Other patient information may also be recorded such as genetic predispositions, family history, lifestyle factors and other information.

A third operation 516 may involve combining recorded patient measurements and medical history to form a comorbidity score.

A fourth operation 518 may involve measuring the patient's BP as SBP and DBP using a commercially available and/or conventional brachial cuff or some other measurement means.

A fifth operation 520 may involve measuring the patient's pulse waveforms. The pulse waveforms may be measured using a modified BP cuff in accordance with the foregoing embodiments. For example, a modified blood pressure cuff that inflates to specific pressures and holds those pressures to capture a waveform at the set pressure may be used. Hold pressures may include DBP, SBP, MAP, and/or sSBP. In some embodiments, an sSBP, which completely cuts off the flow of blood through an artery, may be used. The inflation pressure may be, for example, about 100 mmHg. The hold time may be about, for example, 40 seconds.

Sub operation 522 to the fifth operation 520 may involve performing the pulse waveform measurements for a duration long enough to account for pressure amplitude fluctuations throughout a breathing cycle. Measured amplitudes may be highest at the post-exhalation stage of the breathing cycle.

A sixth operation 524 may include calibrating the measured pulse waveform(s) using the SBP and DBP measurements. The waveform may be calibrated with pressure units utilizing BP measurement results. Calibration methods may include the methods disclosed in the following section of this disclosure.

A seventh operation 526 may include selecting a plurality of post-exhalation waveforms for feature extraction. Post-exhalation waveforms may be selected because these waveforms may track the highest LVEDP reading throughout a breathing cycle. Extracted features and/or parameters of interest may include augmentation index (“AIX”), systolic pulse area, and/or systolic BP. Other desirable and/or relevant features and/or parameters may also be extracted.

An eighth operation 528 may include measuring and/or extracting the features and/or parameters of interest in the pulse waveforms. A sub operation 530 to the eight operation 528 may involve extracting SBP or systolic pulse area. A sub operation 532 to the eighth operation 528 may involve extracting the AIX.

In some embodiments, a classification algorithm may be used to assess risk. Inputs for the algorithm may be pulse features of systolic pulse area, augmentation index and patient features of weight and comorbidity score. Using the inputs and algorithm, a probability of having LVEDP greater than or equal to a failure threshold may be generated. In an embodiment, the failure threshold may be set at about 18 mmHg. In another embodiment, the threshold may be set at about 15 mmHg or at another value of clinical relevance selected when training the algorithm. This process may be repeated for post-exhalation pulses in n breathing cycles to generate n probability predictions. A plurality of measurements may be taken. For example, in an embodiment, two or three measurement may be taken. The probability of the plurality of individual pulses may be combined into a single risk prediction using ensemble methodologies. The predictions may be processed together to generate a single LVEDP risk prediction. Ensemble methodologies may include averaging the probabilities and/or may include more complex methods of aggregating the probabilities.

Accordingly, a ninth operation 534 may involve inputting selected features and/or parameters for each pulse to predict individual LVEDP risk. The selected features and/or parameters may include the systolic pulse area, AIX, patient weight, and comorbidity score. Other parameters and/or combinations of parameters are also possible. Likewise, a tenth operation 536 may involve combining individual pulse risk predictions for patient LVEDP risk prediction.

### Calibration Embodiment

BP cuff measurements may serve as useful clinical measurements because peripheral BP, as measured via a cuff, tends to track central BP in healthy patients. Unfortunately, in patients experiencing cardiovascular issues, the relationship between peripheral BP and central BP may degrade. The severity of the cardiovascular issues in a patient may affect the extent to which the peripheral-central BP relationship degrades. However, because BP cuff measurements are quick, non-invasive, and inexpensive to perform, BP cuff measurements remain an important diagnostic tool for patients experiencing cardiovascular issues. Though the peripheral-central BP relationship degrades in such patients, calibration methods may allow a peripheral BP measurement performed with, for example, a brachial BP cuff, to serve as a proxy for a central BP measurement even in a patient experiencing severe issues.

A modified BP cuff system, such as systems described in the foregoing embodiments, may be used to measure peripheral BP. Peripheral BP may be measured over several breathing holds cycles due to pressure fluctuations caused by breathing. A non-invasive pulse signal measured using a modified BP cuff system may be calibrated to track central BP magnitudes in a patient.

An envelope function may be used to correct a peripheral BP measurement and calibrate a signal. The envelope function may comprise a relationship between pulse amplitude and cuff pressure at measurement site. The measurement cite may be an artery. An envelope function may be constructed by measuring pulse amplitude corresponding to cuff pressure across multiple breath holds.

A calibration method may include several operations. A first operation may involve measuring peripheral BP in the form of systolic BP (“SBP”), diastolic BP (“DBP”), and mean arterial pressure (“MAP”) using a conventional and/or commercially available oscillometric cuff. These measured values may not be accurate. Specifically, these measured values may not track measurement taken in vivo due to amplitude fluctuations caused by breathing and/or due to other errors.

A second operation may involve using a modified BP cuff. The modified BP cuff may be a modified BP system including high resolution pressure sensors and high range pressure sensors, as described in the foregoing embodiments. The second operation may involve inflating the modified BP cuff to a set pressure value. Pulsations may be recorded at that pressure value. The BP cuff may be inflated over a range of set pressure values. Pulsations may be recorded for each pressure value. The second operation may involve taking operations over multiple breath holds to account for pressure fluctuations caused by breathing. For each breath hold, the measured waveform may be analyzed to compare signal pulse amplitude to the set pressure of the modified BP cuff. Measurements may be taken over a plurality of holds to reconstruct a proxy of the envelope function. For example, measurements may be taken over two, three, or more breath holds.

A third operation may involve using the measurements taken during the second operation to calculate parameters to correct for pressure fluctuations due to breathing changes and ultimately to calibrate peripheral measurements to central measurements. This may be accomplished by using an envelope function to derive the parameters needed to correct the peripheral measurements.

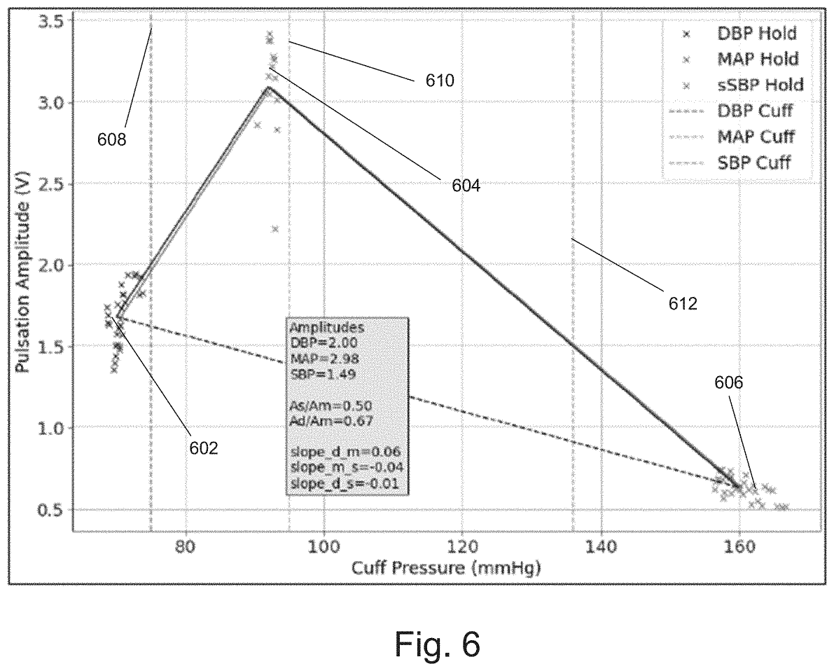

FIG. 6 shows an example of an envelope function reconstructed with a BP measurement and three pressure holds. The three pressure holds are DBP, MAP, and suprasystolic BP (“sSBP”). For the DBP pressure hold, the modified BP cuff was inflated with minimal pressure, allowing blood to flow through the artery substantially unobstructed. For the MAP pressure hold, the modified BP cuff was inflated to MAP. For the sSBP hold, the modified pressure cuff was inflated above the pressure for SBP to effectively cut off any blood flow through the artery. Cuff pressure is shown in FIG. 6 as dashed vertical lines for the three hold values. The three holds values include the DBP hold 608, the MAP hold 610, and the sSBP hold 612.

FIG. 6 also shows individual record pulsations measured at each hold pressure. FIG. 6 shows individual pulsations 602 for the DBP hold, individual pulsations 604 for the MAP hold, and individual pulsations 606 for the sSBP hold. As shown in the example of FIG. 6, multiple pulsations may be recorded for each pressure hold. The pulse amplitudes of the individual pulsations 602, 604, 606 may be measured with a pressure sensor. In the example shown in FIG. 6, pulsation amplitude is reported in volts (V). These pulse amplitude measurements may also be converted into other pressure units.

As discussed above, breathing may cause substantial fluctuations in central BP. The voltage-based signals from a cuff pressure hold may show breathing fluctuations just as in a catheter aortic signal. Therefore, BP values reported by a modified BP cuff measurement may be assumed to be mean values. Pulse signals may be calibrated to pressure units by adjusting SBP and DBP values over a breath pattern and correctly scaling the measured pressure signal. For each individual pulsation in a segment of pulsations, the pulse amplitude difference from the mean pulse amplitude of the segment may be used to correct the SBP and/or DBP values for breathing fluctuations.

For example, a model to correct DBP values from peripheral to central DBP using the envelope function derived parameters is given by:

$${DBP}_{corr} = {DBP}_{cuff} + {m}_{1} * \frac{{A}_{d}}{{A}_{m}} + {m}_{2} * {MAP}_{cuff} + b$$

where

$$\frac{{A}_{d}}{{A}_{m}}$$

is the ratio between the pulse amplitude at DBP versus MAP and DBP_{cuff} and MAP_{cuff} are the DBP and MAP reported by the cuff BP reading respectively, and m₁, m₂, and b are coefficients optimized for the correlation.

SBP values may be corrected using forward and reflect wave peaks measurable in a pulse waveform signal. For the brachial cuff, a potential hold pressure that shows these features is sSBP. Corrected SBP may be given by:

SBP_{corr}=SBP_{cuff}+m₁*(P₁−P₂)+b

where SBP_{corr} is the corrected SBP value to track central BP, SBP_{cuff} is the SBP cuff measurement readout, P₁ is the peak pressure of the first peak in systole, P₂ is the peak pressure of the second peak in systole, and m₁ and b are coefficients optimized for the correlation.

A linear envelope function model may be used to calculate the actual pressure as shown in the following close form equation:

$${P}_{adj} = {P}_{calib} + \frac{Δ PA}{{slope}_{p}}$$

where P_{adj} is the breathing adjusted pressure, P_{calib} is the BP reported value, ΔPA is the pulse amplitude difference from mean, and slopeₚ is the envelope function slope for the specific pressure (slope_{DBP} or slope_{SBP}). Pressure may be either SBP or DBP.

In an uncalibrated segment of pulsations, repeating the foregoing calculations for every pulsation in the segment of pulsations and utilizing signal scaling methodologies, all pulsations can be calibrated with SBP and DBP values that reflect breathing patterns. The model presented assumes a linear relationship between measurement points and a fixed envelope function for a given subject. With more hold pressures, a more detailed envelope function may be reconstructed and may increase model accuracy. Calibration may be applied to SBP and/or DBP independently.

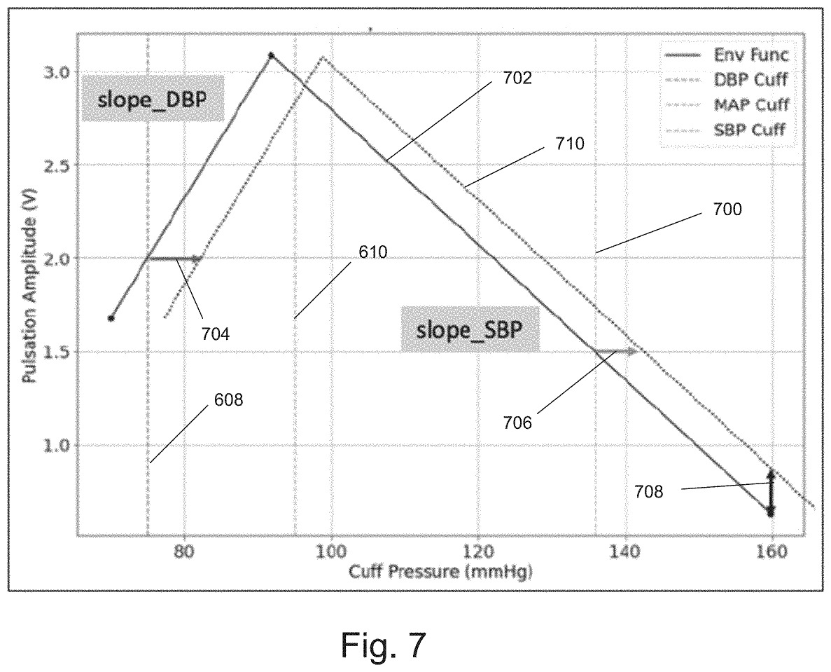

FIG. 7 shows an example of an envelope function used to estimate SBP and DBP changes following pulse amplitude fluctuations. As in FIG. 6, pressure cuff holds are represented by dashed vertical lines. FIG. 7 shows a DBP pressure cuff hold value 608, a MAP pressure cuff hold value 610, and a SBP pressure cuff hold value 700. The arrow 708 at the bottom right of FIG. 7 shows the pulse amplitude deviation from mean pulse amplitude. The arrow 706 above and right of the arrow 708 shows the SBP increase. The arrow 704 at the left of FIG. 7 shows the DBP increase. The envelope function 702 can then be used to estimate SBP and DBP changes following fluctuations in pulse amplitude, which may be caused by breathing. FIG. 7 shows the estimate 710. FIG. 7 is exemplary only. The methodology described above with reference to FIG. 7 may be performed with different devices, threshold conditions, and quantities if the necessary information described is obtained.

In some embodiments, a calibration method incorporating breathing fluctuations may also serve as a diagnostic tool in cardiology. For example, the condition of pulsus paradoxus is defined as a fall in SBP greater than about 10 mmHg during inspiration. This condition may be observed during cardiac tamponade or right ventricle distension such as in severe acute asthma or chronic obstructive pulmonary disease. Therefore, an embodiment of a calibration method may involve setting a threshold of about 10 mmHg.

It should be understood that the various features, aspects and functionality described in one or more of the individual embodiments are not limited in their applicability to the particular embodiment with which they are described. Instead, they can be applied, alone or in various combinations, to one or more other embodiments, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. The terms “a” or “an” should be read as meaning “at least one,” “one or more” or the like; and adjectives such as “conventional,” “traditional,” “normal,” “standard,” “known.” Terms of similar meaning should not be construed as limiting the item described to a given time period or to an item available as of a given time. Instead, they should be read to encompass conventional, traditional, normal, or standard technologies that may be available or known now or at any time in the future. Where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the aspects or functionality described or claimed as part of the component are all configured in a common package. Indeed, any or all of the various aspects of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

The terms “substantially,” “approximately,” and “about” are used throughout this disclosure, including the claims, are used to describe and account for small fluctuations, such as due to variations in processing. For example, they can refer to less than or equal to±5%, such as less than or equal to±2%, such as less than or equal to±1%, such as less than or equal to±0.5%, such as less than or equal to±0.2%, such as less than or equal to±0.1%, such as less than or equal to±0.05%.

What is claimed is:

1. A non-invasive pressure pulse waveform measurement system comprising:
a blood pressure cuff;
an air pump to inflate the blood pressure cuff to specific pressure levels;
high resolution pressure sensors configured to perform high sensitivity signal acquisition at a specified pressure level, wherein each high resolution pressure sensor includes a measurement port and a reference port;
high range pressure sensors configured to measure an absolute reference for the signal and to calibrate the signal;
pneumatic tubing connecting the air pump and sensors with the cuff; and
a hydrodynamic filter configured as an input to the reference port of each high resolution pressure sensor;
wherein the hydrodynamic filter is configured to transmit only mean pressure by attenuating a selected frequency range of the signal.

2. The system of claim Error! Reference source not found, wherein the hydrodynamic filter comprises a resistive component and a capacitance component.

3. The system of claim 2, wherein the resistive component of the hydrodynamic filter is configured to impose a resistance to flow.

4. The system of claim 2, wherein the capacitive component is configured to reduce pressure changes by storing air volume.

5. The system of claim 2, wherein the capacitance component comprises tubing that connects the resistive component to the reference port.

6. The system of claim 2, wherein the resistive component of the hydrodynamic filter comprises rigid tubing with an internal diameter in the range of 10-200 μm.

7. The system of claim 4, wherein an elasticity of the capacitive element is in the range of 0.2-2.0 MPa.

8. The system of claim 1, wherein the blood pressure cuff is synchronized with an ECG device, iPhone, tablet, computer, or other device, thereby allowing the transmission of wired or wireless data.

9. The system of claim 8, wherein Bluetooth or Wi-Fi is employed for the transmission of wireless data.

10. The system of claim 2, wherein the resistive component and the capacitive component are combined within a single component.

11. The system of claim 2, wherein the resistive component comprises resistive elements including an orifice or a physical filter.

12. The system of claim 11, wherein the orifice is fixed and/or adjustable.

13. The system of claim 2, wherein the capacitive component comprises a compliant elastic tube, a tube having a small damper, or a tube having a piston cylinder.
